(19) United States
(12) Patent Application Publication
(10) Pub. No.: US 2017/0315310 A1
(43) Pub. Date: Nov. 2, 2017

(54) OPTICAL TRANSMISSION MODULE AND ENDOSCOPE

(71) Applicant: OLYMPUS CORPORATION, Tokyo (JP)

(72) Inventor: Yusuke NAKAGAWA, Kamiina-gun (JP)

(73) Assignee: OLYMPUS CORPORATION, Tokyo (JP)

(21) Appl. No.: 15/650,074

(22) Filed: Jul. 14, 2017

Related U.S. Application Data
(63) Continuation of application No. PCT/JP2015/051881, filed on Jan. 23, 2015.

(51) Int. Cl. G02B 6/42; A61B 1/00; G02B 23/24; A61B 1/07

(52) U.S. Cl. CPC G02B 6/42; A61B 1/00096; G02B 23/2469; A61B 1/00013; A61B 1/07

(57) ABSTRACT

An optical transmission module includes an optical element, an optical fiber, a holding section to which the optical fiber is bonded and fixed, and a wiring board including a first principal plane on which the optical element is mounted and a second principal plane on which the holding section is bonded and a connection pad connected to the optical element is disposed, and through which light passes. An adhesive tape is inserted between the holding section and the wiring board. The reinforcing resin does not protrude to an outside of the detachment sheet on the second principal plane of the wiring board.

### CROSS REFERENCE TO RELATED APPLICATION

This application is a continuation application of PCT/JP2015/051881 filed on Jan. 23, 2015, the entire contents of which are incorporated herein by this reference.

### BACKGROUND OF THE INVENTION

### 1. Field of the Invention

The present invention relates to an optical transmission module including an optical element and an optical fiber for transmitting light of an optical signal outputted by a light emitting section of the optical element and an endoscope including the light transmission module.

### 2. Description of the Related Art

An endoscope includes an image pickup device such as a CCD at a distal end portion of an elongated insertion section. In recent years, it has been examined to use, in an endoscope, an image pickup device having a large number of pixels. When the image pickup device having a large number of pixels is used, a signal volume transmitted from the image pickup device to a signal processing device (a processor) increases. Therefore, optical signal transmission via a thin optical fiber by an optical signal using an optical transmission module is desirable instead of electric signal transmission via a metal wire by an electric signal.

For example, as disclosed in Japanese Patent Application Laid-Open Publication No. 2013-025092, an optical transmission module is manufactured by bonding a wiring board on which an optical element is surface-mounted and an optical-fiber holding section (a ferrule) into which an optical fiber is inserted.

### SUMMARY OF THE INVENTION

An optical transmission module of an embodiment of the present invention is an optical transmission module including: an optical element including, on a light emitting surface, a light emitting section configured to output light of an optical signal; an optical fiber for transmitting the optical signal; a holding section to which the optical fiber is bonded and fixed such that light outputted by the light emitting section is made incident on an end face of the optical fiber; and a wiring board which includes a first principal plane on which the optical element is mounted and a second principal plane on which the holding section is bonded and a connection pad connected to the optical element is disposed, and through which the light outputted by the light emitting section passes. A part of the optical fiber and a whole of the holding section are covered by reinforcing resin. A detachment sheet is inserted between the holding section and the wiring board. The reinforcing resin does not protrude to an outside of the detachment sheet on the second principal plane of the wiring board.

An endoscope of another embodiment is an endoscope including an optical transmission module provided at a distal end portion of an insertion section. The optical transmission module includes: an optical element including, on a light emitting surface, a light emitting section configured to output light of an optical signal; an optical fiber for transmitting the optical signal; a holding section to which the optical fiber is bonded and fixed such that light outputted by the light emitting section is made incident on an end face of the optical fiber; and a wiring board which includes a first principal plane on which the optical element is mounted and a second principal plane on which the holding section is bonded and a connection pad connected to the optical element is disposed, and through which the light outputted by the light emitting section passes. A part of the optical fiber and a whole of the holding section are covered by reinforcing resin. A detachment sheet is inserted between the holding section and the wiring board. The reinforcing resin does not protrude to an outside of the detachment sheet on the second principal plane of the wiring board.

### BRIEF DESCRIPTION OF THE DRAWINGS

FIG. 1 is a sectional view of an optical transmission module of a first embodiment;

FIG. 2 is a top view of the optical transmission module of the first embodiment;

FIG. 3 is a sectional view for explaining a manufacturing method for the optical transmission module of the first embodiment;

FIG. 4 is a sectional view for explaining a recycling method for the optical transmission module according to the first embodiment;

FIG. 5 is a top view of an adhesive tape of the optical transmission module of the first embodiment;

FIG. 6 is a sectional view of an optical transmission module of a second embodiment;

FIG. 7 is a sectional view for explaining a manufacturing method for an optical transmission module of a third embodiment;

FIG. 8 is a sectional view of the optical transmission module of the third embodiment;

FIG. 9 is a top view of an adhesive tape of an optical transmission module of a fourth embodiment;

FIG. 10 is a top view of an adhesive tape of an optical transmission module of a fifth embodiment;

FIG. 11 is a sectional view of an optical transmission module of a sixth embodiment;

FIG. 12 is a sectional view of an optical transmission module of a seventh embodiment;

FIG. 13 is a sectional view of an optical transmission module of a modification of the seventh embodiment;

FIG. 14 is a sectional view of an optical transmission module of a modification of the seventh embodiment; and

FIG. 15 is a perspective view of an endoscope of an eighth embodiment.

### DETAILED DESCRIPTION OF THE PREFERRED EMBODIMENTS

### First Embodiment

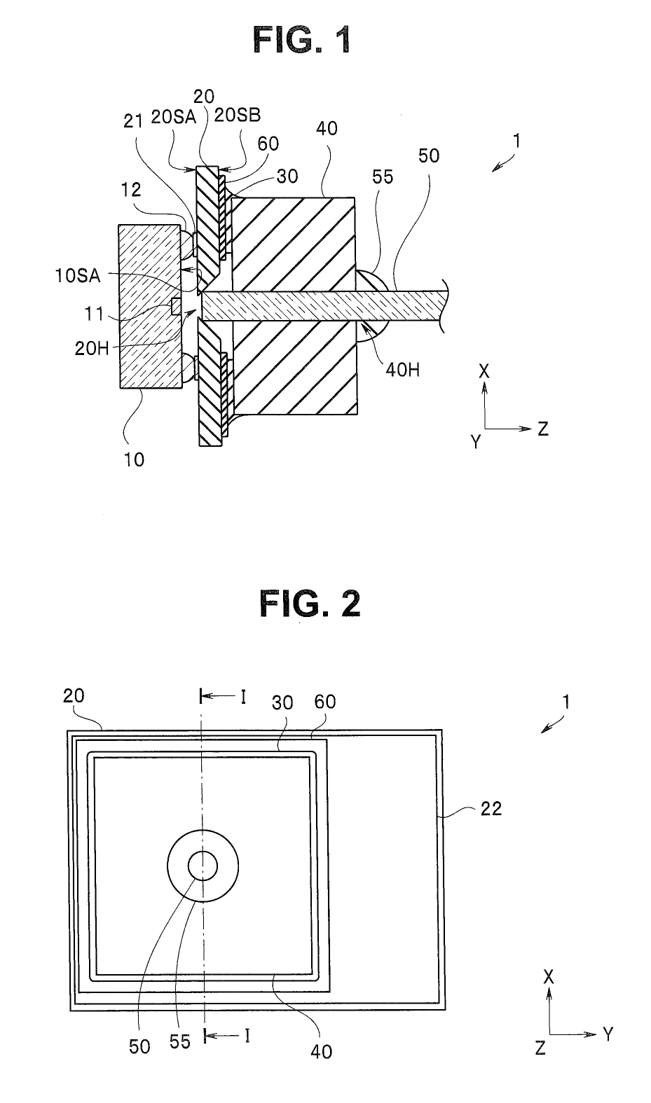

As shown in FIG. 1 and FIG. 2, an optical transmission module 1 of the present embodiment includes an optical element 10, a wiring board 20, a holding section (a ferrule) 40, and an optical fiber 50. In the optical transmission module 1, the optical element 10, the wiring board 20, and the holding section 40 are disposed side by side in a thickness direction of the optical element 10 (a Z direction). Note that FIG. 1 is a sectional view taken along (a I-I line) in FIG. 2.

Note that all of the drawings are schematic and relations between thicknesses and widths of respective portions, ratios of the thicknesses of the respective portions, and the like are different from real ones. Relations and ratios of dimensions of which are different from one another, are included in some portions among the drawings.

The optical element 10 is a VCSEL (vertical cavity surface emitting laser) including, on a light emitting surface 10SA, a light emitting section 11 configured to output light of an optical signal. For example, the optical element 10 of an ultra-small type having a plan view dimension of 250 μm×300 μm includes, on the light emitting surface 10SA, the light emitting section 11 having a diameter of 20 μm and electrodes 12 configured to supply a driving signal to the light emitting section 11.

On the other hand, for example, the optical fiber 50 is an easily aligned MMF (multi mode fiber). A core for transmitting light has a diameter of 50 μm and a clad covering an outer circumference of the core has a diameter of 125 μm.

A distal end portion of the optical fiber 50 is inserted into a through-hole 40H of the holding section 40 having a substantially rectangular parallelepiped shape bonded on the optical element 10 and is fixed by an adhesive 55. The optical fiber 50 is inserted into the through-hole 40H to be fixed in a position on which light outputted by the light emitting section 11 is made incident.

A hole 20H, through which the light outputted by the light emitting section 11 passes, is present in the wiring board 20 having a flat shape including a first principal plane 20SA and a second principal plane 20SB. The optical element 10 is flip-chip mounted on the first principal plane 20SA in a state in which the light emitting section 11 is disposed in a position opposed to the hole 20H of the wiring board 20. That is, the wiring board 20 includes, on the first principal plane 20SA, a plurality of electrodes 12 of the optical element 10 and electrode pads 21 to which the electrodes 12 are respectively bonded. On the other hand, the wiring board 20 includes, on the second principal plane 20SB, a connection pad 22 (see FIG. 2) such as a solid GND connected to the electrode pads 21 via a through-wire or the like. As a substrate of the wiring board 20, a resin substrate, a ceramic substrate, a glass epoxy substrate, a glass substrate, a silicon substrate, or the like is used. Note that the wiring board 20 is particularly desirably an FPC (flexible printed circuit) board including polyimide or the like as a substrate from viewpoints of a reduction in size and flexibility.

For example, Au bumps, which are the electrodes 12 of the optical element 10, are ultrasound-bonded to the electrode pads 21 of the wiring board 20. Note that an adhesive such as an under-fill material or a side-fill material may be injected into a bonding section.

After solder paste or the like is printed on the wiring board 20 and the optical element 10 is disposed in a predetermined position, solder may be melted by reflow or the like to mount the optical element 10. Note that the wiring board 20 may include a processing circuit for converting an electric signal transmitted from an image pickup device 90 into a driving signal of the optical element 10.

As explained above, the through-hole 40H having a columnar shape having an inner diameter substantially the same as an outer diameter of the optical fiber 50 inserted into the through-hole 40H is formed in the holding section 40. “Substantially the same” means that diameters of the optical fiber 50 and the through-hole 40H are substantially “same” sizes that bring an outer circumferential surface of the optical fiber 50 and a wall surface of the through-hole 40H into a contact state. For example, the inner diameter of the through-hole 40H is formed larger by 1 μm to 5 μm with respect to the outer diameter of the optical fiber 50.

The through-hole 40H may have a prism shape besides the columnar shape as long as the optical fiber 50 can be held by the wall surface of the through-hole 40H. A material of the holding section 40 is a metal member or the like such as ceramic, Si, glass, or SUS. Note that the holding section 40 may have a substantially columnar shape, a substantially conical shape, or the like.

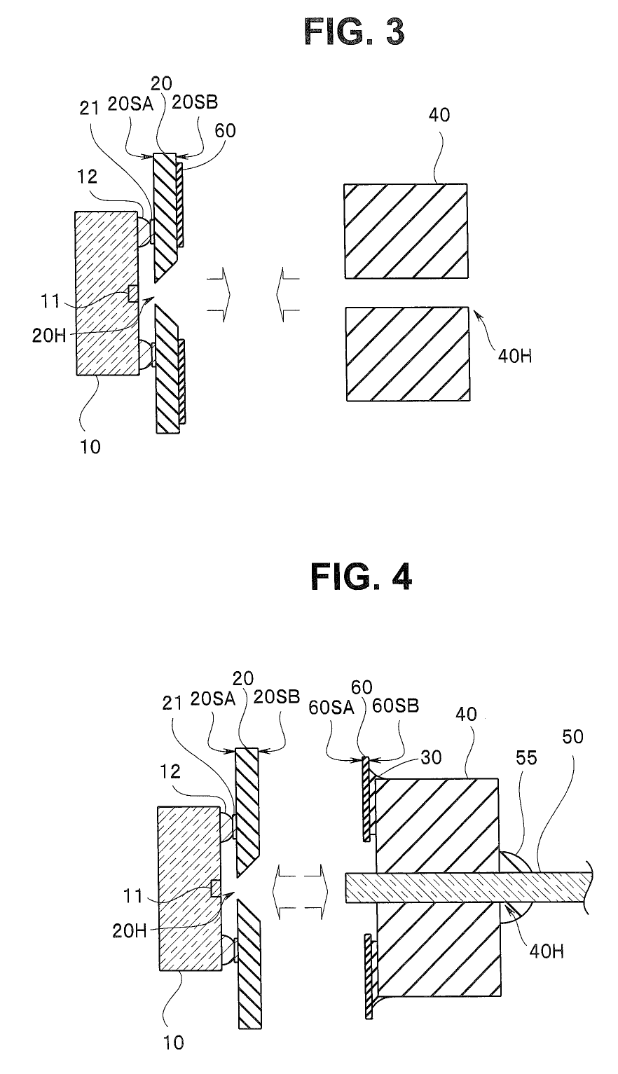

As shown in FIG. 3, the optical transmission module 1 is manufactured by bonding the wiring board 20 on which the optical element 10 is mounted and the holding section 40.

In the hole 20H of the wiring board 20, an opening of the second principal plane 20SB is larger than an opening of the first principal plane 20SA, the opening of the second principal plane 20SB is larger than a diameter of the optical fiber 50, and the opening of the first principal plane 20SA is smaller than the diameter of the optical fiber 50.

After the wiring board 20 and the holding section 40 are bonded, the optical fiber 50 is inserted such that a distal end face comes into contact with the wall surface of the hole 20H of the wiring board 20. By using the wiring board 20 including the hole 20H having a tapered shape, a distance between a light emitting surface of the optical element 10 and an end face of the optical fiber 50 can be managed short and accurate. Therefore, it is possible to improve coupling efficiency. The above is explanation in the case in which the hole 20H is present in the wiring board 20. However, even when the hole 20H is absent in the wiring board 20, the wiring board 20 can be used as the optical transmission module 1 by using a substrate having transmittance to a wavelength of VCSEL light as the wiring board 20 and abutting the optical fiber 50 against the second principal plane of the wiring board 20. However, concerning the coupling efficiency, a form in which the hole 20H is formed is considered to be advantageous.

In the optical transmission module 1, in the holding section 40, an adhesive surface of an adhesive tape 60, which is a detachment sheet (a detachment member), is stuck to the wiring board 20. Thereafter, the holding section 40 is bonded to the wiring board 20 by an adhesive 30 via the adhesive tape 60.

The adhesive 30 formed of ultraviolet curing resin or thermosetting resin firmly fixes the adhesive tape 60 and the holding section 40. Note that, in order to secure bonding strength, an outer circumferential section of the adhesive 30 is desirably formed in a fillet shape as shown in FIG. 1.

On the other hand, the adhesive tape 60 is, for example, a polyimide film, on one surface of which an adhesive layer is disposed. Bonding strength of the adhesive tape 60 measured under conditions of (a detachment angle of 90 degrees and detachment speed of 50 mm/min) conforming to JIS Z 0237 is 6 N/25 mm.

In a manufacturing process of an optical transmission module and an assembly process for assembling the optical transmission module in a housing or the like, tensile stress and compression stress are sometimes applied to an optical fiber. If applied with excessive stress or if greatly bent, the optical fiber is sometimes broken. The optical transmission module made defective because, for example, the optical fiber is damaged is discarded. This is a cause of an increase in cost of the optical transmission module.

In the manufacturing process and the like of the optical transmission module, production of defective products is unavoidable. However, as shown in FIG. 4, the optical transmission module 1 can be easily detached between the holding section 40 attached with the optical fiber 50 and the wiring board 20 attached with the optical element 10.

More specifically, the adhesive tape 60 can be completely peeled by pinching an end portion of the adhesive tape 60 with a sharpened tool such as tweezers and detaching the adhesive tape 60 while gripping the adhesive tape 60.

Therefore, it is possible to easily recycle the wiring board 20 attached with the optical element 10 of the optical transmission module made defective because the optical fiber 50 is broken. The wiring board 20 attached with the optical element 10 to be recycled is used with the adhesive tape 60 stuck to the wiring board 20 again.

In the optical transmission module 1, the expensive optical element 10 made defective and discarded can be recycled. Therefore, it is possible to achieve a cost reduction of the optical transmission module 1.

Note that the recycle process is a special process performed only when a defective product is produced. Therefore, the wiring board 20 and the holding section 40 may be directly bonded only by the adhesive 30 not via the adhesive tape 60. Note that, in this case, the optical transmission module including the recycled wiring board 20 attached with the optical element 10 has a configuration same as a configuration of a conventional optical transmission module not including the adhesive tape 60. However, when a manufactured plurality of optical transmission modules include an optical transmission module including the adhesive tape 60 and an optical transmission module not including the adhesive tape 60, it is seen that the optical transmission module not including the adhesive tape 60 is a recycled produced.

In the optical transmission module 1, a distal end face of the optical fiber 50 is brought into contact with the wall surface of the taper-shaped hole 20H of the wiring board 20, whereby the distance between the light emitting surface of the optical element 10 and the end face of the optical fiber 50 is determined. Therefore, even in the optical transmission module not including the adhesive tape 60, the coupling efficiency is not deteriorated.

At least during the manufacturing of the optical transmission module 1, the adhesive tape 60 needs to stably hold the holding section 40 and the wiring board 20. During the recycling, the adhesive tape 60 needs to be able to be detached. Therefore, bonding strength of the adhesive tape 60 is desirably 1 N/25 mm or more and 15 N/25 mm or less and particularly desirably 5 N/25 mm or more and 8 N/25 mm or less.

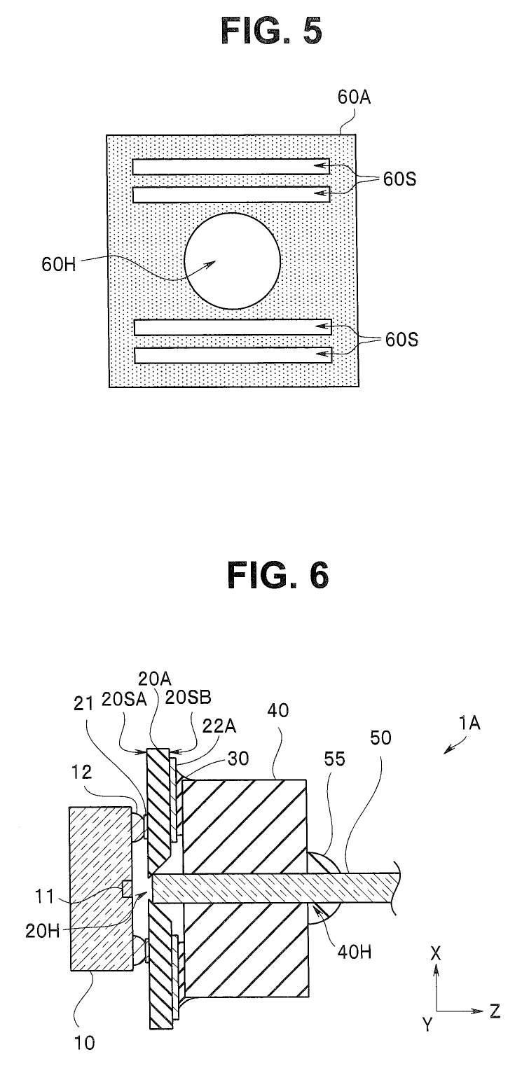

In order to improve the bonding strength, holes may be provided in the adhesive tape 60. For example, an adhesive tape 60A shown in FIG. 5 includes not only a hole 60H functioning as an optical path but also slit-like four holes 60S. In portions of the holes 60S, the holding section 40 and the wiring board 20 are directly bonded via only the adhesive 30. Therefore, the holding section 40 and the wiring board 20 are more firmly bonded than when all bonding surfaces are bonded via the adhesive tape 60. A region bonded via only the adhesive 30 is small. Therefore, when the adhesive tape 60A is detached, the region bonded via only the adhesive 30 can also be detached. A size, a shape, the number of pieces, and the like of the hole 6011 are determined according to specifications of the optical transmission module.

As a detachment sheet that stably holds the holding section 40 and the wiring board 20 during the manufacturing and can be detached during the recycling, a so-called thermal detachment adhesive sheet, bonding strength of which greatly decreases when heat is applied, may be used instead of the adhesive tape 60. In this case, the bonding strength after the heating only has to be within the range.

In order to make it easy to pinch the adhesive tape 60 with a sharpened tool such as tweezers when the adhesive tape 60 is detached, a convex portion more convex than other portions may be formed in at least a part of an end portion. For example, the convex portion is formed by detaching an adhesive layer of the end portion of the adhesive tape 60 in advance or inserting and sticking a small film between the end portion of the adhesive tape 60 and the wiring board 20. Note that a convex portion formed in a frame shape along an outer peripheral portion of the adhesive tape 60 has action of a bank (a dam) for preventing the adhesive 30 from flowing out to an outside of the adhesive tape 60.

### Second Embodiment

An optical transmission module 1A of a second embodiment is explained. Note that all of optical transmission modules of embodiments and optical transmission modules of modifications explained below are similar to the optical transmission module 1 of the first embodiment. Therefore, components having the same functions are denoted by the same reference numerals and signs and explanation of the components is omitted.

As shown in FIG. 6, in the optical transmission module 1A of the present embodiment, a metal pattern 22A, which is a detachment sheet, is disposed on the second principal plane 20SB of a wiring board 20A. The metal pattern 22A is made of a material same as a material of the connection pad 22 such as the solid GND of the wiring board 20A, for example, copper. That is, in a manufacturing process of the wiring board 20A, when the connection pad 22 is manufactured, the metal pattern 22A is simultaneously manufactured by, for example, etching treatment of copper foil or a copper plating process. That is, the metal pattern 22A and the connection pad 22 are made of the same material.

The holding section 40 is bonded to the wiring board 20A via the metal pattern 22A and the adhesive 30 on the metal pattern 22A. Note that, since the metal pattern 22A is a part of the wiring board 20A, in a more strict sense, the holding section 40 is bonded by the adhesive 30 via the metal pattern 22A of the wiring board 20A.

Bonding strength of the metal pattern 22A to a substrate is desirably 1 N/25 mm or more and 15 N/25 mm or less and particularly desirably 5 N/25 mm or more and 8 N/25 mm or less like the bonding strength of the adhesive tape 60 explained above.

In the optical transmission module 1A, as in the optical transmission module 1, the expensive optical element 10 can be recycled. Therefore, it is possible to achieve a cost reduction of the optical transmission module 1A.

Further, in the optical transmission module 1A, the metal pattern 22A, which is the detachment sheet, can be formed simultaneously with wires, electrodes, and the like of the wiring board 20. Therefore, the optical transmission module 1A can be manufactured in a simpler process and more inexpensively than the optical transmission module 1.

Note that, in the optical transmission module 1A, as in the optical transmission module 1, by using the wiring board 20A including the taper-shaped hole 20H, the distance between the light emitting surface of the optical element 10 and the end face of the optical fiber 50 can be managed short and accurate. Therefore, it is possible to improve the coupling efficiency.

In the optical transmission module 1A, as in the optical transmission module 1, in order to make it easy to pinch an end portion of the metal pattern 22A with a sharpened tool such as tweezers when the metal pattern 22A is detached, a convex portion may be formed. For example, the convex portion can be formed by providing, for example, a solder resist pattern in advance in a region of the second principal plane 20SB to be formed as an end portion of the metal pattern 22A and disposing the metal pattern 22A on the solder resist pattern.

Note that, in a recycling process of the optical transmission module 1A, the wiring board 20 and the holding section 40 are directly bonded. Note that, therefore, the optical transmission module including the recycled wiring board 20 attached with the optical element 10 has a configuration same as the configuration of the conventional optical transmission module. However, when a manufactured plurality of optical transmission modules include an optical transmission module including the adhesive tape 60 and an optical transmission module not including the adhesive tape 60, it is seen that the optical transmission module not including the adhesive tape 60 is a recycled produced.

### Third Embodiment

An optical transmission module 1B of a third embodiment is explained.

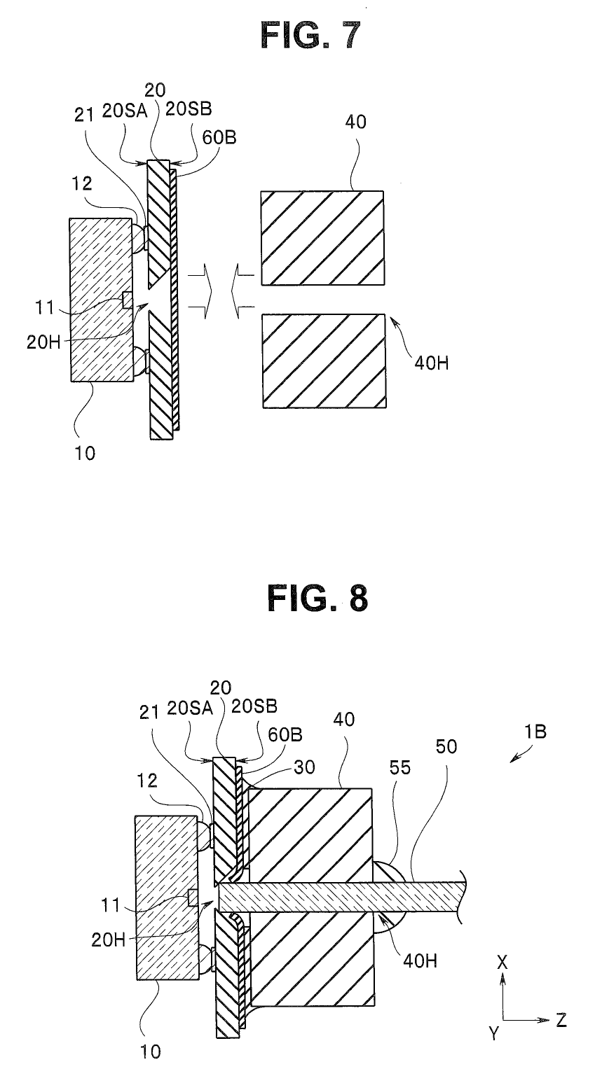

As shown in FIG. 7, the optical transmission module 1B includes an adhesive tape 60B substantially the same as the adhesive tape 60 of the optical transmission module 1. However, a hole for allowing insertion of the optical fiber 50 is not formed in the adhesive tape 60B in advance. However, as shown in FIG. 8, the optical fiber 50 penetrates through the adhesive tape 60B and comes into contact with the wall surface of the hole 2011 of the wiring board 20.

That is, in a manufacturing process of the optical transmission module 1B, the optical fiber 50 is inserted into the through-hole 40H of the holding section 40 and penetrates through the adhesive tape 60B, whereby a hole is formed in the adhesive tape 60B.

Note that thickness of the adhesive tape 60B is selected such that the optical fiber 50 can penetrate through the adhesive tape 60B. In the adhesive tape 60B, at least thickness of a portion through which the optical fiber 50 penetrates only has to be thickness through which the optical fiber 50 can penetrate. A substrate material of the adhesive tape 60B is, for example, polyimide.

When the adhesive 30 is applied, the adhesive 30 sometimes intrudes into a taper of the hole 20H of the wiring board 20. Then, it is likely that a shape of the wall surface changes and the adhesive 30 flows into the optical element 10.

In the optical transmission module 1B, even if the adhesive 30 reaches an upper part of the taper of the hole 20H, since the adhesive tape 60B does not have a hole, it is unlikely that the adhesive 30 flows into the optical element 10. Therefore, the optical transmission module 1B has an effect same as the effect of the optical transmission module 1 and the like. Further, a manufacturing yield is high.

### Fourth Embodiment, Fifth Embodiment

An optical transmission module 1C of a fourth embodiment and an optical transmission module 1D of a fifth embodiment are explained.

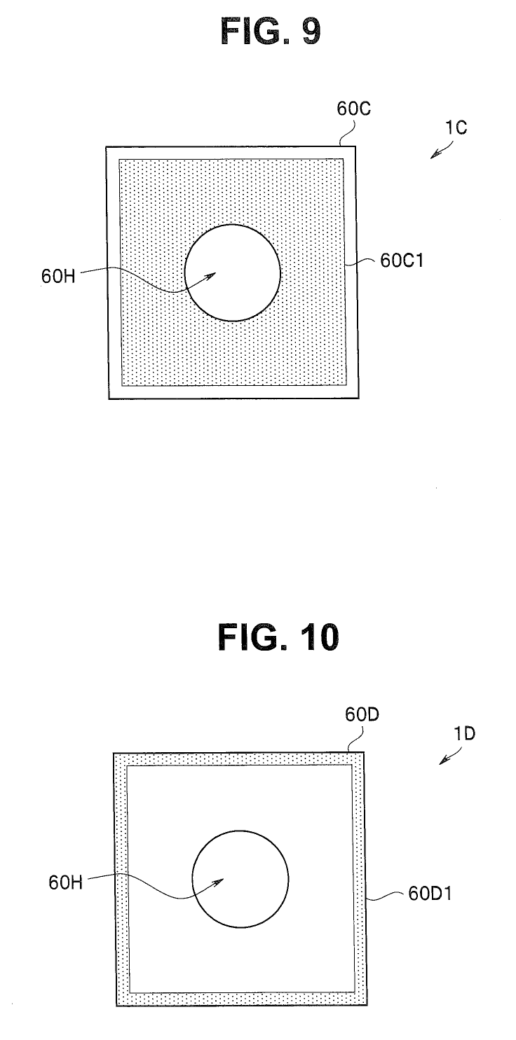

As shown in FIG. 9, in an adhesive tape 60C of the optical transmission module 1C, a region 60C1 where the adhesive 30 of the adhesive tape 60 is applied is subjected to hydrophilic treatment. On the other hand, as shown in FIG. 10, in an adhesive tape 60D of the optical transmission module 1D, a region 60D1 where the adhesive 30 of the adhesive tape 60 is not applied is subjected to hydrophobic treatment.

The adhesive 30 is in a hydrophilic liquid state before hardening.

Therefore, in the adhesive tape 60C and the adhesive tape 60D, the adhesive 30 does not spread exceeding a desired range of the adhesive tape 60C and the adhesive tape 60D.

Therefore, the optical transmission modules 1C and 1D have the effect of the optical transmission module 1. Further, the adhesive tape 60C and the adhesive tape 60D can be surely detached.

### Sixth Embodiment

An optical transmission module 1E of a sixth embodiment is explained.

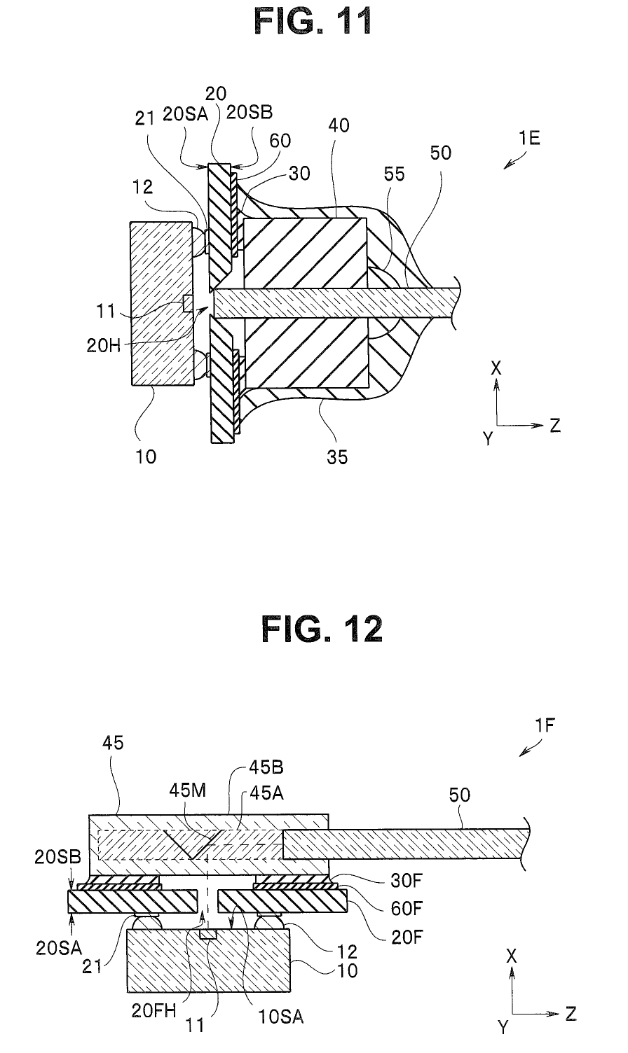

As shown in FIG. 11, in the optical transmission module 1E, potting resin (POT resin) 35, which is reinforcing resin, is heaped high on the second principal plane 20SB of the wiring board 20 to cover the entire holding section 40 and a part of the optical fiber 50. The POT resin covers the adhesive 30 as well but is in contact with the wiring board 20 via the adhesive tape 60.

For example, the POT resin 35 made of thermosetting resin is disposed after the optical fiber 50 is provisionally fixed in the holding section 40 by the adhesive 55. The POT resin 35 is applied not to protrude from the adhesive tape 60 stuck to the wiring board 20 and is subjected to hardening treatment.

The POT resin 35 increases mechanical strength between the optical fiber 50 and the holding section 40 and mechanical strength between the holding section 40 and the adhesive tape 60. Note that the POT resin 35 heaped high on the wiring board 20 has not only a reinforcement effect but also a moisture proof improvement effect.

By peeling a detachment sheet, the holding section 40, on which the POT resin 35 is heaped, can also be easily detached from the wiring board 20 together with the optical fiber 50. That is, the optical transmission module 1E has an effect same as the effect of the optical transmission module 1 and the like.

Note that, as the optical transmission module 1E, a form is shown in which the POT resin 35 is disposed in the optical transmission module 1. However, in the optical transmission modules 1A to 1D, it is desirable to dispose the POT resin 35 as in the optical transmission module 1.

### Seventh Embodiment

An optical transmission module 1F of a seventh embodiment is explained.

As shown in FIG. 12, in the optical transmission module 1F, the optical fiber 50 is disposed in parallel to the light emitting surface 10SA of the optical element 10. Note that the optical transmission module 1 and the like in which the optical fiber 50 is disposed perpendicularly to the light emitting surface 10SA are referred to as “vertical installation”. The optical transmission module 1F is referred to as “horizontal installation”. In the optical transmission module 1F, the optical fiber 50 is disposed in a rib structure of an optical waveguide substrate 45 and fixed by an adhesive (not shown in the figure). That is, the optical waveguide substrate 45 has a holding section function, an optical element function for bending an optical path by 90 degrees, and a light transmission function.

The optical transmission module 1 and the like are configured to directly couple the light generated by the optical element 10 to the optical fiber 50. On the other hand, the optical transmission module 1F couples the light generated by the optical element 10 to the optical fiber 50 via the optical waveguide substrate 45.

In the optical waveguide substrate 45, as disclosed in, for example, Japanese Patent Application Laid-Open Publication No. 2012-113180, a core 45A is made of polymer having a refractive index n1, a clad 42B is made of polymer having a refractive index n2, and n1>n2. In the optical waveguide substrate 45, a mirror 45M for bending an optical path by 90 degrees is formed.

The rib structure for disposing the optical fiber 50 in a predetermined position is present on an optical path exit side of the optical waveguide substrate 45. The rib structure includes two parallel protrusion sections. Note that a connecting direction of the optical fiber 50 to the optical waveguide substrate 45 may be either a substrate horizontal direction or a substrate vertical direction. However, from viewpoints of manufacturing easiness and positioning accuracy improvement, the substrate horizontal direction that makes use of the rib structure as in the optical transmission module 1F is desirable.

A manufacturing method for the optical transmission module 1F is briefly explained.

First, an adhesive tape 60F is stuck to the second principal plane 20SB of a wiring board 20F. Note that a hole 20FH of the wiring board 20F is not formed in a tapered shape. Note that, when a thin plate made of light transmissive resin or the like is used as the wiring board 20F not to hinder passage of light emitted by the optical element 10, that is, when transmittance in output light from a light emitting element such as a VCSEL is high, the hole 20FH is unnecessary.

The optical waveguide substrate 45 is bonded to the second principal plane 20SB of the wiring board 20F by an adhesive 30F via the adhesive tape 60F. Further, the optical element 10 is surface-mounted on the first principal plane 20SA of the wiring board 20F.

The optical fiber 50 is inserted into the rib structure of the optical waveguide substrate 45 and fixed by an adhesive (not shown in the figure).

The optical transmission module 1F includes the optical element 10 including the light emitting section 11 configured to output light of an optical signal, the optical fiber 50 for transmitting the optical signal, the optical waveguide substrate 45 equivalent to the holding section configured to fix the end face of the optical fiber 50 in a position on which the light outputted by the light emitting section 11 is made incident, and the wiring board 20F including the first principal plane 20SA on which the optical element 10 is mounted and the second principal plane 20SB to which the optical waveguide substrate 45 is bonded, the light outputted by the light emitting section 11 passing through the wiring board 20F. The optical transmission module 1F is detachable between the optical waveguide substrate 45 and the wiring board 20F.

That is, the optical waveguide substrate 45, which is the holding section, is bonded to the wiring board 20F via the adhesive tape 60F bonded to the wiring board 20F and the adhesive 30F on the adhesive tape 60F. Like the adhesive tape 60, bonding strength of the adhesive tape 60F is desirably 1 N/25 mm or more and 15 N/25 mm or less and particularly desirably 5 N/25 mm or more and 8 N/25 mm or less.

In the optical transmission module 1F, as in the optical transmission module 1, the expensive optical element 10 can be recycled. Therefore, it is possible to achieve a cost reduction of the optical transmission module 1F. Further, in the optical transmission module 1F, since the rib structure of the optical waveguide substrate 45 is used, positioning of the optical fiber 50 is easy. Thickness (in the X direction) of the optical transmission module 1F is smaller than the thickness of the optical transmission module 1 and the like. Since the light generated by the optical element 10 can be coupled to the optical fiber 50 with a low coupling loss by the optical waveguide substrate 45, a stable transmission characteristic can be obtained.

Note that, in the optical transmission module 1F, the metal pattern of the wiring board 20F may be used as the detachment sheet instead of the adhesive tape 60F as in the optical transmission module 1A.

Even various optical transmission modules of the “horizontal installation” type have an effect same as the effect of the present invention if a detachment sheet is inserted between a holding section to which an optical fiber is fixed and a wiring board on which an optical element is mounted.

For example, a silicon substrate or the like including a rib structure or a V groove, which can fix and bond an optical fiber, an end face of which is machined into a 45-degree mirror, in a region of the wiring board to which the detachment sheet is stuck can be used as the holding section.

Further, detachment may be facilitated by bonding the holding section and the optical fiber via the detachment sheet. When the optical fiber is broken, the optical element can be recycled by detaching only the optical fiber from the optical transmission module and bonding a new optical fiber.

When the holding section configured to fix the optical fiber has a function of the wiring board including electrode pads or the like on which the optical element is mounted, naturally, either one of the wiring board or the holding section is unnecessary. For example, in the case of an optical transmission module of the horizontal installation type including a silicon substrate, which is a wiring board and is a holding section including electrode pads or the like on which the optical element is mounted, the optical fiber being bonded and fixed to the silicon substrate such that light outputted by the light emitting section is made incident on the end face of the optical fiber, the detachment sheet is disposed in a V groove that fixes the optical fiber.

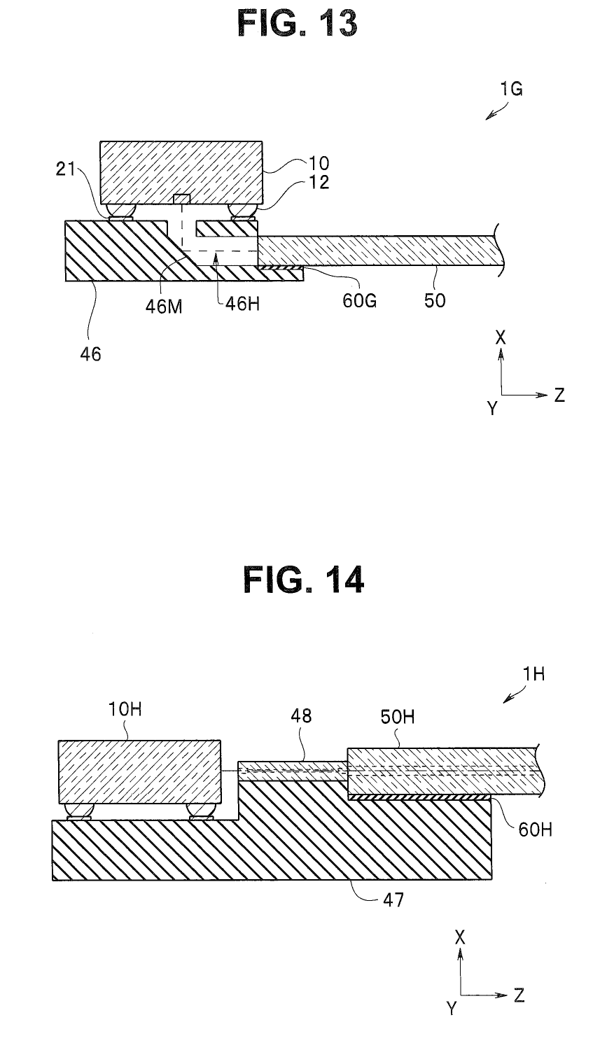

In an optical transmission module 1G of a modification of the seventh embodiment shown in FIG. 13, an adhesive tape 60G is stuck to a V groove of the silicon substrate 46 including a through-hole 46H functioning as a waveguide on an inside, an end face of the through-hole 4611 being a 45-degree reflection mirror 46M. The optical fiber 50 is bonded and fixed via the adhesive tape 60G. Note that an inner surface of the through-hole 46H is desirably covered with a reflection film because it is possible to improve transmission efficiency.

In an optical transmission module 1H of a modification of the seventh embodiment shown in FIG. 14, light of an optical element 10H, which emits light from a side surface, is made incident on a single-mode optical fiber 50H via a silicon photonics waveguide 48. An adhesive tape 60H is stuck to a V groove of a silicon substrate 47, which is a wiring board and is a holding section. The optical fiber 5011 is bonded and fixed via the adhesive tape 60H. Note that a size converter (not shown in the figure) made of SiN is manufactured on an end face of the silicon photonics waveguide 48.

Note that, when an optical fiber is fixed to a V groove of a silicon substrate by an Si compound instead of the adhesive tape 60G, it is possible to detach the optical fiber by etching and removing the Si compound with hydrofluoric acid or the like.

### Eighth Embodiment

An endoscope 2 of an eighth embodiment is explained. The endoscope 2 includes the optical transmission module 1 or 1A to 1H (“optical transmission module 1 or the like”) explained above in a rigid distal end portion 81 of an insertion section 80.

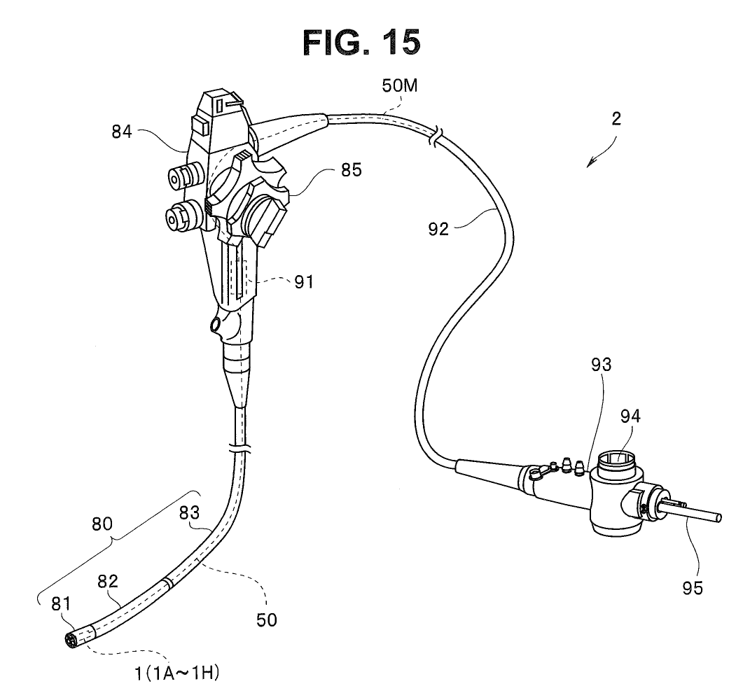

As shown in FIG. 15, the endoscope 2 includes the insertion section 80, an operation section 84 disposed on a proximal end portion side of the insertion section 80, a universal cord 92 extended from the operation section 84, and a connector 93 disposed on a proximal end portion side of the universal cord 92.

In the insertion section 80, the rigid distal end portion 81, a bending section 82 for changing a direction of the rigid distal end portion 81, and an elongated flexible portion 83 are jointly provided in order.

In the endoscope 2, an image pickup signal is converted into an optical signal by the optical transmission module 1 or the like, which is an E/O module of the rigid distal end portion 81, and transmitted to the operation section 84 via the thin optical fiber 50 inserted through the insertion section 80. The optical signal is converted into an electric signal again by an O/E module 91 disposed in the operation section 84 and transmitted to an electric connector section 94 via a metal wire 50M inserted through the universal cord 92. That is, a signal is transmitted via the optical fiber 50 in the insertion section 80 having a small diameter. The signal is transmitted via the metal wire 50M thicker than the optical fiber 50 in the universal cord 92 that is not inserted into a body and has less limitation on an outer diameter.

Note that, when the 0/E module 91 is disposed in the electric connector section 94, the optical fiber 50 may be inserted through the universal cord 92 to the electric connector section 94. When the 0/E module 91 is disposed in a processor, the optical fiber 50 may be inserted through to the connector 93.

In the operation section 84, an angle knob 85 for operating the bending section 82 is disposed and the 0/E module 91, which is an optical transmission module that converts an optical signal into an electric signal, is disposed. The connector 93 includes the electric connector section 94 connected to a processor (not shown in the figure) and a light-guide connecting section 95 connected to a light source. The light-guide connecting section 95 is connected to an optical fiber bundle configured to guide illumination light to the rigid distal end portion 81. Note that, in the connector 93, the electric connector section 94 and the light-guide connecting section 95 may be integrated.

The optical transmission module 1 or 1A to 1H is small in size, in particular, small in diameter. Therefore, the endoscope 2 including the optical transmission module 1 or 1A to 1H is small in diameter in a distal end portion and an insertion section. Therefore, the endoscope 2 is less invasive.

In the optical transmission module 1 or 1A to 1H, even if a defective product is produced, the expensive optical element 10 can be recycled. Therefore, it is possible to achieve a cost reduction of the endoscope 2.

The present invention is not limited to the embodiments explained above. Various changes, combinations, and applications may be made within a range not departing from the spirit of the invention.

What is claimed is:

1. An optical transmission module comprising:
an optical element including, on a light emitting surface, a light emitting section configured to output light of an optical signal;
an optical fiber for transmitting the optical signal;
a holding section to which the optical fiber is bonded and fixed such that light outputted by the light emitting section is made incident on an end face of the optical fiber; and
a wiring board which includes a first principal plane on which the optical element is mounted and a second principal plane on which the holding section is bonded and a connection pad connected to the optical element is disposed, and through which the light outputted by the light emitting section passes, wherein
a part of the optical fiber and a whole of the holding section are covered by reinforcing resin,
a detachment sheet is inserted between the holding section and the wiring board, and
the reinforcing resin does not protrude to an outside of the detachment sheet on the second principal plane of the wiring board.

2. The optical transmission module according to claim 1, wherein bonding strength between the holding section and the wiring board is in a range of 1 N/25 mm or more and 15 N/25 mm or less.

3. The optical transmission module according to claim 2, wherein
the holding section is bonded to the wiring board via an adhesive tape, which is the detachment sheet bonded to the wiring board, and an adhesive on the adhesive tape, and
the bonding strength of the adhesive tape is in the range.

4. The optical transmission module according to claim 2, wherein
the holding section is bonded to the wiring board via a metal pattern made of a material same as a material of the connection pad, the metal pattern being the detachment sheet disposed on the second principal plane of the wiring board, and an adhesive on the metal pattern, and
the bonding strength of the metal pattern is in the range.

5. The optical transmission module according to claim 1, wherein
a through-hole is present in the holding section,
the optical fiber is inserted through the through-hole,
a hole through which the light outputted by the light emitting section passes is present in the wiring board, and
the hole is formed in a tapered shape in which an opening in the first principal plane of the hole is smaller than a diameter of the optical fiber and an opening in the second principal plane of the hole is larger than the diameter of the optical fiber, and
an end face of the optical fiber is in contact with a wall surface of the hole of the wiring board.

6. The optical transmission module according to claim 1, wherein the optical fiber penetrates through the detachment sheet.

7. The optical transmission module according to claim 3, wherein
the adhesive is in a hydrophilic liquid state before hardening, and
a region of the detachment sheet, the region being applied with the adhesive is subjected to hydrophilic treatment or a region not applied with the adhesive is subjected to hydrophobic treatment.

8. The optical transmission module according to claim 1, wherein at least a part of an end portion of the detachment sheet is convex with respect to another part.

9. The optical transmission module according to claim 1, wherein the optical fiber is disposed perpendicularly to the light emitting surface.

10. The optical transmission module according to claim 1, wherein the optical fiber is disposed in parallel to the light emitting surface.

11. An endoscope comprising the optical transmission module according to claim 1 provided at a distal end portion of an insertion section.
